(19) United States
(12) Patent Application Publication
(10) Pub. No.: US 2008/0188718 A1
(43) Pub. Date: Aug. 7, 2008

(54) SURGICAL RETRACTOR WITH ADJUSTABLE BLADES AND METHOD OF USE

(76) Inventors: James Spitler, Plano, TX (US); Noelle Dye, Charlestown, MA (US)

Correspondence Address: CARR LLP (IST), 670 FOUNDERS SQUARE, 900 JACKSON STREET, DALLAS, TX, 75202, US

(21) Appl. No.: 12/021,100

(22) Filed: Jan. 28, 2008

Related U.S. Application Data
(60) Provisional application No. 60/886,704, filed on Jan. 26, 2007.

(51) Int. Cl. A61B 1/32

(52) U.S. Cl. 600/213; 600/215; 600/232

(57) ABSTRACT

A surgical retractor may be provided for a surgical procedure such as spinal surgery. The surgical retractor may include a separator, a first and second blade holder coupled to the separator, and blade assemblies coupled to the blade holders. The separator may move the blade holders to change the position of the first blade holder relative to the second blade holder. The blade assemblies may retract tissue of a patient. An activator of a blade holder may be used to rotate a first blade of the blade assembly. Rotating the first blade tilts the blade assembly to create a larger opening at a distal end of the retractor. In some embodiments, side blades may be placed in the retractor.

### PRIORITY CLAIM AND RELATED APPLICATIONS

This application claims priority to U.S. Provisional Patent Application 60/886,704 entitled “SURGICAL RETRACTOR DEVICE AND METHOD OF USE” to Spitler et al. filed Jan. 26, 2007, which is incorporated by reference in its entirety. This application is related to U.S. Utility application Ser. No. ______, entitled “SURGICAL RETRACTOR WITH REPLACEABLE BLADES AND METHOD OF USE” (10-004-US2) to Boucher, et al., filed on the same date as this application, which is also incorporated by reference in its entirety for all purposes.

### BACKGROUND

1. Field of the Invention

The present invention relates generally to surgical retractors. More particularly, the invention relates to a surgical retractor for minimally invasive procedures with blade assemblies that allow for a larger opening at a distal end of the retractor than at a proximal end of the retractor.

2. Description of Related Art

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae (typically twenty-four) separated by intervertebral discs. The intervertebral discs hold the vertebrae together in a flexible manner so as to allow relative movement between the vertebrae from front to back and from side to side. This movement allows the body to bend forward and backward, to bend from side to side, and to rotate about a vertical axis. When the spine is operating properly, the nerves are maintained clear of the hard structure of the spine throughout the available ranges of motion.

Over time or because of accidents or disease, the intervertebral discs may lose height or become cracked, dehydrated, or herniated. The result is that the height of one or more discs may be reduced. The reduction in height can lead to compression of the nerve bundles. Such compression may cause pain and, in some cases, damage to the nerves.

Currently, there are many systems and methods at the disposal of a physician for reducing or eliminating the pain by minimizing the stress on the nerve bundles. In some instances, the existing disc is removed and an artificial disc is substituted therefore. In other instances, two or more vertebrae are fused together to prevent relative movement between the fused discs.

In some procedures, minimally invasive surgical procedures have been developed to fuse or otherwise treat vertebrae. Such procedures can reduce pain, post-operative recovery time, and the destruction of healthy tissue. Minimally invasive surgical procedures are particularly desirable for spinal and neurosurgical applications because of the need for access to locations deep within the body and the possible range of damage to vital intervening tissues.

Generally, it is desirable to access the surgical site using minimally invasive techniques or portals, rather than through a significant incision, to aid in preserving the integrity of the intervening tissues. In such procedures, however, it may be necessary to hold the edges of an incision apart to provide a clear operating field within which the surgeon can operate.

What is needed, therefore, is a tool or retractor adapted to work with minimally invasive procedures that allows the surgeon to have a clear path to the operating field, and a method for using such a tool or retractor.

### SUMMARY

Disclosed herein are various embodiments described related to a surgical retractor. In certain embodiments, the surgical retractor includes a first blade holder and a second blade holder. The first blade holder includes an activator. A first blade assembly is positionable in the first blade holder. The first blade assembly includes at least a first blade and a second blade. A second blade assembly is positionable in the second blade holder. The activator of the first blade holder is configured to rotate the first blade assembly relative to the first blade holder to tilt the first blade assembly and create a larger opening at a distal end of the surgical retractor. In some embodiments, the first blade assembly is positioned in a blade holder. The blade holder may be rotated relative to the separator.

In some embodiments, an illumination source may be coupled to the first blade assembly. In some embodiments, a length of the blades is adjustable.

In some embodiments, the first blade holder and the second blade holder are coupled to a separator. The separator may be configured to move the first blade holder relative to the second blade holder. In certain embodiments, the separator may comprise a bar, a first rack coupled to the bar, a second rack coupled to the bar, and a gear coupled to the bar, the first rack and the second rack. The gear is configured to move the first rack relative to the second rack to move the first blade holder relative to the second blade holder. In some embodiments, the separator includes a stop coupled to the gear. The stop is removably coupled to the first rack. Movement of the first blade holder relative to the second holder is inhibited until the stop is decoupled from the first rack.

In some embodiments, the first blade holder is removably coupled to the separator, and the first blade assembly is affixed to the first blade holder. In some embodiments, the first blade holder is affixed to the separator, and the first blade assembly is removably coupled first blade holder.

In other embodiments, there is disclosed various methods of retraction during surgery. In some embodiments, the methods include placing a first and second blade assembly of a surgical retractor in an opening in a patient. A separator is activated to move the first blade assembly away from the second blade assembly and retract tissue. A first blade of the first blade assembly is rotated to tilt the first blade assembly and obtain additional tissue retraction with a larger opening located at the distal end of the retractor. In some embodiments, a first blade of the second blade assembly is rotated to tilt the second blade assembly and obtain additional tissue retraction with a larger opening located at the distal end of the retractor. In some embodiments, one or more side blades are inserted in the surgical retractor.

In some embodiments described herein, a kit may be provided for a surgical procedure. The kit may include a separator, at least a pair of blade holders configured to couple to the separator, at least a pair of blade assemblies configured to couple to the blade holders, and at least one driver configured to activate one or more moving components of the separator and the blade holders. The kit may also include at least a pair of side blades, a blade adjustor configured to lengthen a portion of a blade assembly, at least one illumination source configured to couple to a blade assembly, and/or a dilator set.

### BRIEF DESCRIPTION OF THE DRAWINGS

Features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts a perspective view of an embodiment of a surgical retractor.

FIG. 2 depicts a perspective view of an embodiment of a separator of a surgical retractor that emphasizes a top of the separator.

FIG. 3 depicts a perspective view of an embodiment of a separator of a surgical retractor that emphasizes a bottom of the separator.

FIG. 4 depicts a perspective view of an embodiment of a blade holder of a surgical retractor.

FIG. 5 depicts a perspective view of an embodiment of an attachment piece of a blade holder.

FIG. 7 depicts a perspective view of an embodiment of an angle lock of a blade holder.

FIG. 8 depicts a perspective view of an extender of a first blade of a surgical retractor.

FIG. 9 depicts a perspective view of an embodiment of a blade assembly without extenders.

FIG. 10 depicts a front view of an embodiment of a first blade of a blade assembly.

FIG. 11 depicts a top view of an embodiment of a first blade of a blade assembly.

FIG. 12 depicts a front view of an embodiment of a second blade of a blade assembly.

FIG. 13 depicts a top view of an embodiment of a second blade of a blade assembly.

FIG. 14 depicts a perspective view of an embodiment of a surgical retractor with side blades inserted in the blade holders of the surgical retractor.

FIG. 15 depicts a perspective view of an embodiment of a side blade for a surgical retractor.

FIG. 16 depicts a side view of an embodiment of a side blade for a surgical retractor.

FIG. 17 depicts a top view of one embodiment of a surgical kit.

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF EMBODIMENTS

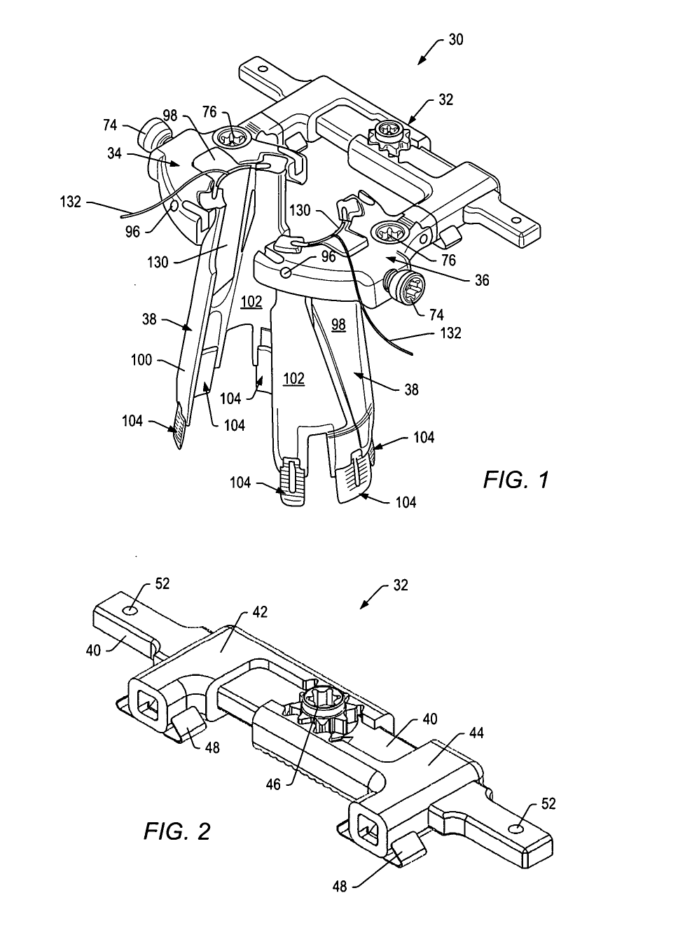

FIG. 1 depicts an embodiment of a surgical retractor 30. Surgical retractor 30 may include separator 32, first blade holder 34, second blade holder 36, and blade assemblies 38. Surgical retractor 30 may be used during a surgical procedure to retract tissue of a patient to provide a surgeon access to an operating field. In some embodiments, surgical retractor 30 is used during spinal surgery. Surgical retractor 30 may also be used during other types of surgical procedures. Surgical retractor 30 may allow for a relatively small opening to be formed in the patient to accommodate needed access to the surgical site during the surgical procedure. When blade assemblies 38 are positioned in the opening, separator 32 may be used to expand the opening by moving first and second blade holders 34, 36 away from each other. Separator 32 inhibits undesired movement of first blade holder 34 relative to second blade holder 36 so that retracted tissue does not result in the first blade holder being forced towards the second blade holder with a resulting decrease in the size of the opening. The opening in the patient at the distal end of blade assemblies 38 may be enlarged by rotating or tilting the blade assemblies so that the area of retractor tissue is larger at the distal end of the surgical retractor than near blade holders 34, 36. Providing a larger area at the distal end of the surgical retractor may advantageously provide better visualization of the operating field.

Separator 32 of surgical retractor 30 may allow for relative movement of first blade holder 34 towards or away from second blade holder 36. In some embodiments, first blade holder 34 moves the same distance but in an opposite direction relative to second blade holder 36. Movement of first blade holder 34 relative to the second blade holder 36 the same distance but in an opposite direction allows for equal expansion of blades assemblies 38 relative to a midline between the blade holders.

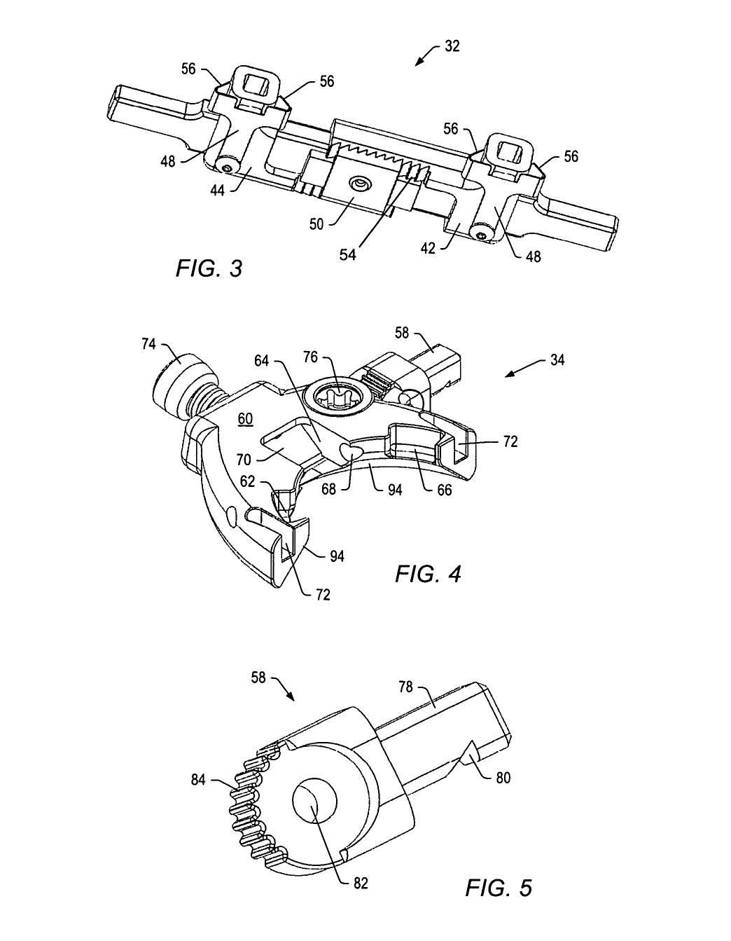

In some embodiments, separator 32 is a rack and pinion type of mechanism. FIG. 2 and FIG. 3 depict an embodiment of separator 32. Separator 32 may include bar 40, first rack 42, second rack 44, pinion 46, couplers 48 and stop 50 (shown in FIG. 3). Bar 40 may include recesses 52 that allow the surgical retractor to be coupled to the surgical table to stabilize and fix the position of the surgical retractor relative to the patient. In other embodiments, bar 40 or other portions of the surgical retractor may include connection features that allow the surgical retractor to be coupled to the surgical table such as, but not limited to recesses, threaded openings, protrusions, grooves, slots, and/or quick release mechanisms.

First rack 42 and second rack 44 may slide along bar 40. First rack 42 and second rack 44 may include gear teeth that engage gear teeth of pinion 46. Pushing pinion 46 downwards and rotating the pinion moves first rack 42 towards or away from second rack 44. Indicia placed on, printed on or etched in bar 40 and/or racks 42, 44 may indicate direction of travel of first rack and/or second rack when pinion 46 is rotated in a clockwise direction or counterclockwise direction. In other embodiments, separator 32 includes a threaded shaft and a wheel or other type of activator that allows for movement of the first blade holder of the surgical retractor relative to the second blade holder of the surgical retractor. Other systems for separating the first blade holder from the second blade holder may also be used.

FIG. 3 depicts a perspective view of an embodiment of separator 32 that emphasizes the bottom of the separator. Pushing the pinion of the separator downwards disengages serrations of stop 50 from mating serrations 54 in first rack 42 and/or second rack 44. When the serrations of stop 50 are positioned in the corresponding serrations 54 of first rack 42 and/or second rack 44, movement of the first rack relative to the second rack is inhibited. Stop 50 inhibits undesired movement of first rack 42 relative to second rack 44 so that undesired movement of the blade holders coupled to the separator is inhibited. Stop 50 may inhibit force applied to the blade assemblies by retracted tissue from reducing the size of the opening established by the surgical retractor.

Couplers 48 may be attached to first rack 42 and second rack 44. Couplers 48 may be springs that function to secure blade holders to separator 32. The bias of the springs may force protrusion of couplers 48 into openings in racks 42, 44. The protrusions of couplers 48 may fit in notches in portions of the blade holders to secure the blade holders to separator 32. The front of the protrusions may be angled so that entry of the blade holders into racks 42, 44 moves the protrusions of couplers 48 outwards to facilitate entry of the blade holders into separator 32. Arms 56 of couplers 48 may be moved away from first rack 42 and/or second rack 44 to allow for removal of the blade holders from separator 32. In other embodiments, other fastening systems may be used to couple the blade holders to the separator such as, but not limited to, set screws, spring driven releases, and detents.

FIG. 4 depicts a perspective view of first blade holder 34. The second blade holder may be a mirror image of first blade holder 34. First blade holder 34 may include attachment piece 58, angle lock 76, body 60, cam 70, cam activator 74, first blade recess 62, second blade recess 64, third blade recess 66, pivot axis recess 68, and side blade recesses 72.

FIG. 5 depicts an embodiment of attachment piece 58. End 78 may fit in a recess in the first rack of the separator of the surgical retractor. The protrusion of the coupler of the first rack may fit in notch 80 to secure the first blade holder to the separator. Attachment piece 58 may include opening 82 and teeth 84. The body of the first blade holder may be rotated relative to the separator about a pin positioned in opening 82 when the blade holder is attached to the separator and the angle lock is not in the locked position.

FIG. 1 depicts surgical retractor 30 when separator 32 is in-line with first blade holder 34. FIG. 6 depicts a side view of surgical retractor 30 with angle A established between separator 32 and first blade holder 34 relative to the center of pin 86 that couples attachment piece 58 to body of the first blade holder.

FIG. 5 depicts teeth 84 of attachment piece 58 of a blade holder. Teeth 84 of attachment piece 58 may determine the range of rotation of the blade holder relative to the separator of the surgical retractor. The range of rotation of the separator relative to a blade holder may be from about 130° to about 230°. In other embodiments, the range of rotation of the separator relative to a blade holder may be from about 135° to about 210°, or from about 135° to about 200°. In some embodiments, teeth 84 of attachment pieces 58 allows the angle of the blade holders relative to the separator to be adjusted in 10° increments in the allowable range of rotation. In other embodiments, other increment angles may be selected by adjusting the spacing between teeth 84 of attachment pieces 58. In some embodiments, the attachment piece may include a contact surface instead of teeth. A brake or other contactor of the blade holder may press against the contact surface of the attachment piece when the angle lock is in the locked position to fix the angle between the separator and the blade holder at a desired angle in the available range of rotation.

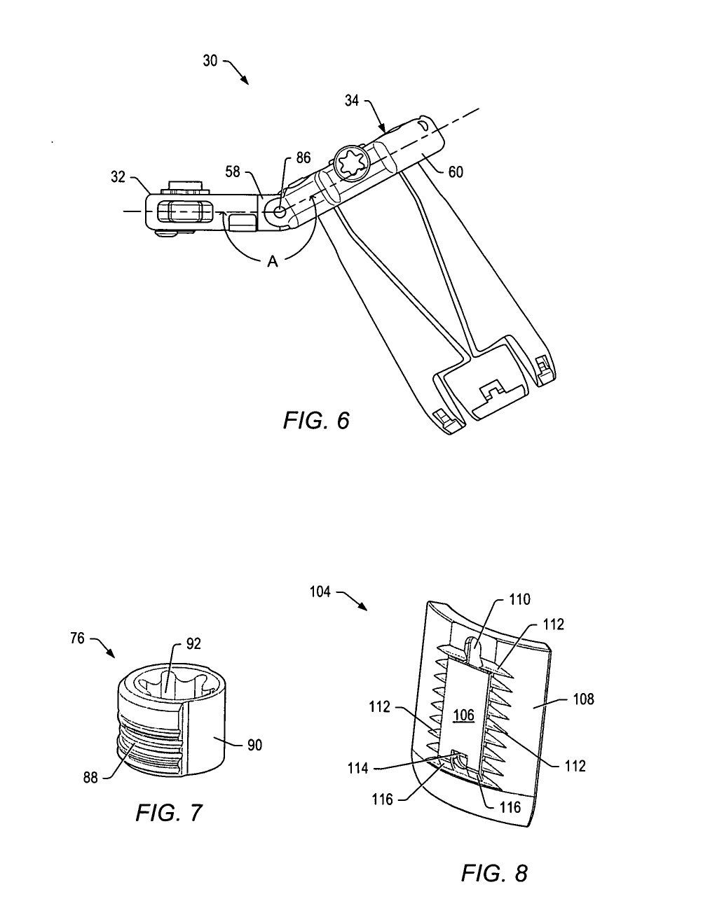

FIG. 7 depicts a perspective view of angle lock 76 separated from the blade holder. Angle lock 76 may include teeth 88, flat portion 90 and tool opening 92. FIG. 4 depicts angle lock 76 positioned in first blade holder 34. A washer or other element may inhibit removal of angle lock 76 from body 60 of first blade holder 34 once the angle lock and washer are positioned in the body. A driver (not shown) with an end that is complementary to tool opening 92 may be used to rotate angle lock relative to body 60 of first blade holder 34. A pin, ledge, and/or other structure in body 60 may interact with angle lock 76 to limit the range of rotation of the angle lock. Indicia printed on, attached to, or etched in body 60 may indicate the rotation direction to lock angle lock 76.

When angle lock 76 is rotated to an open position, flat portion 90 is positioned adjacent to teeth 84 of attachment piece 58 (FIG. 5). In this unlocked position, body 60 may be rotated up or down relative to attachment piece 58 to angle the first blade holder, and the blade assembly positioned in the first blade holder, relative to the separator of the surgical retractor.

When angle lock 76 is rotated to the locked position, teeth 88 of the angle lock are positioned between teeth 84 of attachment piece 58. Positioning teeth 88 of angle lock 76 between teeth 84 of attachment piece 58 inhibits movement of body 60 relative to the attachment piece.

As shown in FIG. 1, first blade holder 34 and second blade holder 36 may include body openings 96. In some embodiments, a connector (not shown) may be coupled to body openings 96 to temporarily couple the blade holders together. Angle locks 76 of each blade holder 34, 36 may be rotated to the open position. The angle of blade holders 34, 36 relative to separator 32 may be adjusted by moving the connector up or down relative to the separator. The connector ensures that first blade holder 34 and second blade holder 36 are at the same angle relative to separator 32. When the desired angle is established, angle locks 76 are rotated to the locked position to inhibit further angle adjustment, and the connector may be removed from blade holders 34, 36.

FIG. 4 depicts cam 70 of first blade holder 34. Cam 70 may be advanced or retracted by turning cam activator 74. In some embodiments, rotating cam activator 74 in a clockwise direction advances cam 70 forward into first blade recess 62. Rotating the activator 74 in a counterclockwise direction moves cam 70 away retracts the cam from first blade recess 62. Cam 70 may contact an angled surface of the first blade positioned in first blade recess 62 to rotate the first blade about a rotation axis. Rotation of the first blade may also rotate the second blade, which is positioned in second blade recess 64; and the third blade, which is positioned in the third blade recess 66. In an embodiment, cam 70 may rotate the blade assembly positioned in blade recesses 62, 64, 66 in an outward direction in a range from an initial position where the first blade is substantially perpendicular to a central axis of first blade holder 34 up to an angle of about 30°. In other embodiments, a smaller or greater range of rotation of the blade assembly may be achieved by the cam. The rotation axis of the first blade may be defined by pins that extend from the first blade and are positioned in pivot axis recesses 68 in body 60. Bottom surface 94 of body 60 may be angled to accommodate rotation of the blade assembly positioned in blade recesses 62, 64, 66.

Body 60 may also include side blade recesses 72. When the separator is activated to move the first blade holder away from the second blade holder, portions of the side blades may be positioned in side blade recesses 72 to inhibit tissue intrusion into the operating field established by the surgical retractor.

FIG. 1 depicts blade assemblies 38 for surgical retractor 30. Blade assemblies 38 may be positioned in first blade holder 34 and second blade holder 36 of surgical retractor 30. Each blade assembly 38 may include a first or main blade 98, a secondary or second blade 100, and another secondary or third blade 102. In some embodiments, second blade 100 is a mirror image of third blade 102.

One or more of blades 98, 100, 102 may include extender 104. Extenders may allow for adjustment of the lengths of blades 98, 100, 102.

FIG. 8 depicts a perspective view of an embodiment of extender 104 for a first blade. Extender 104 may include extender body 108 and ratchet 106. Sides of extender body 108 may fit in a groove in the first blade. Ratchet 106 may be secured to the inner surface of the first blade.

Extender body 108 may include guide 110 and grooves 112. Grooves 112 may be cut in the body to have a sloping upper surface and a substantially vertical bottom surface. Ratchet 106 may include post 114 and arms 116. Post 114 may be positioned in guide 110. Post 114 and guide 110 may limit the travel distance of extender body 108 relative to the first blade. Ratchet arms 116 may be positioned in a groove of grooves 112. When a blade adjustor (not shown) forces extender body 108 downwards relative to first blade, sloping upper surfaces of grooves 112 allows the extension body to move downwards and extend from the body of the first blade. When extender body 108 is moved downwards relative to the body of the first blade, ratchet arms 116 are positioned in a different groove. Retraction of extender body 108 into the body of the first blade is inhibited by contact of ratchet arms 116 with a substantially vertical bottom surface of groove 112. FIG. 1 depicts surgical retractor 30 with extenders 104 pushed out to lengthen the blades of blade assemblies 38. The extender bodies may be retracted into the bodies of the blades after the surgical retractor has been removed from the patient using a tool to disengage the ratchet from the grooves in the extender bodies.

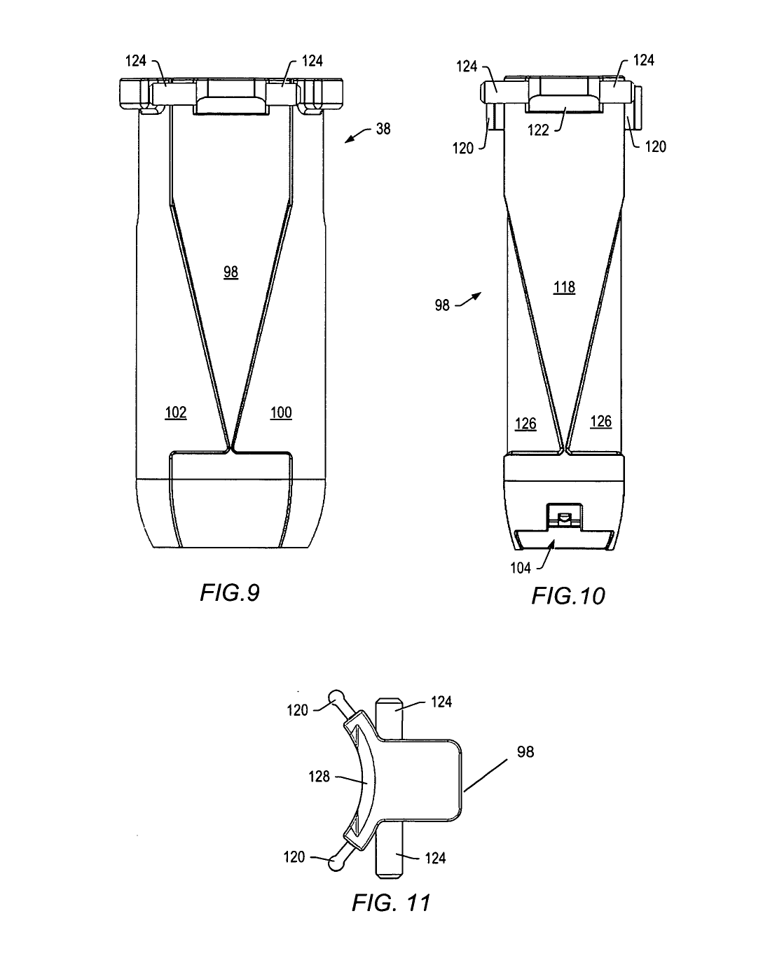

In some embodiments, the ratchet for the second blade and the third blade is identical in size and shape to the ratchet for the first blade. Using the same ratchet for each of the blades limits the number of different parts that need to be formed to produce the surgical retractor. In some embodiments, the extender body of the second blade is identical to the extender body of the third blade. In some embodiments, the extender bodies of the second blade and the third blade are narrower than the extender body of the first blade. In some embodiments, such as the embodiment depicted in FIG. 9, blades 98, 100, 102 of blade assembly 38 do not include extenders.

In some embodiments, the blade assemblies are coupled to blade holders to form blade holder and blade assembly combinations having specific lengths. A number of pairs of blade holder and blade assembly combinations of different lengths may be included in a kit provided for a surgical procedure along with the separator. The blade holder and blade assembly combinations may be etched or printed with indicia that indicate length. A surgeon may select the desired blade holder and blade assembly combination pair and couple the combination pair to the separator.

In some embodiments, the blade assemblies may be insertable and removable from the blade holders during a surgical procedure. A number of blade assembly pairs of various lengths may be included in a kit provided for a surgical procedure along with a pair of blade holders and the separator. Having insertable and removable blade assemblies may significantly reduce the size and weight of the kit provided for the surgical procedure since only a single pair of blade holders is needed and not a pair of blade holders for each pair of blade assemblies. Also, the separator and the blade holders may be formed as a single non-separable unit when insertable and removable blade assemblies are used. Blade assemblies may be positioned in or removed from the blade holders and separator combination as needed.

FIG. 10 depicts a front view of an embodiment of first or main blade 98 of a blade assembly. FIG. 11 depicts a top view of an embodiment of first blade 98. First blade 98 may include body 118, arms 120, cam surface 122, pins 124, and extender 104. An upper portion of body 118 may fit in the first blade recess of a blade holder (not shown). Body 118 may include a raised portion that defines recessed areas 126. The extensions of the second blade and the third blade may be positioned in recessed areas 126 of first blade 98. Arms 120 of first blade 98 may be positioned in recesses of the second blade and the third blade to couple the blade assembly together.

Cam surface 122 may be an angled surface. The cam of a blade holder contacts cam surface 122 when the cam activator of the blade holder is rotated to drive the cam forward. When the cam activator drives the cam forward, the cam contacts cam surface 122 and rotates first blade 98 relative to the blade holder to tilt the first blade.

Pins 124 may be positioned in the pivot axis recesses of a blade holder. In some embodiments, pins 124 are ends of a shaft that is positioned in an opening through body 118 of first blade 98. In some embodiments, pins 124 are press fit into openings formed in body 118 of first blade 98. Pins 124 may be attached to the body of the first blade by other methods, including but not limited to, welding, glue, and/or threading.

As depicted in FIG. 11, the inner surface of the blade may include slot 128. In some embodiments, slot 128 extends about ⅔ of the length of first blade 98. In other embodiments, the slot may extend greater or less than ⅔ of the length of the first blade. Side edges of slot 128 maybe angled. A light mat may slide down into slot 128. The angled side edges may keep the light mat coupled to first blade 98. Light mats may be disposable. Light mats may be available from LumitexMD, Inc., located in Strongsville, Ohio.

In some embodiments, a first end of an optical cable may be coupled to a light source, and a second end of the optical cable may be coupled to the light mat. The light source may provide light to the light mat, and the light mat may illuminate the operating field established by the surgical retractor. If the first blades of the surgical retractor are rotated to tilt the blade assemblies and widen the surgical opening at the distal end of the surgical retractor, the tilt of the first blades of the retractor may allow light provided by light mats coupled to the first blades to illuminate the bottom of the operating field established by the surgical retractor. FIG. 1 depicts light mats 130 and optical cables 132 coupled to first blades 98 of surgical retractor 30.

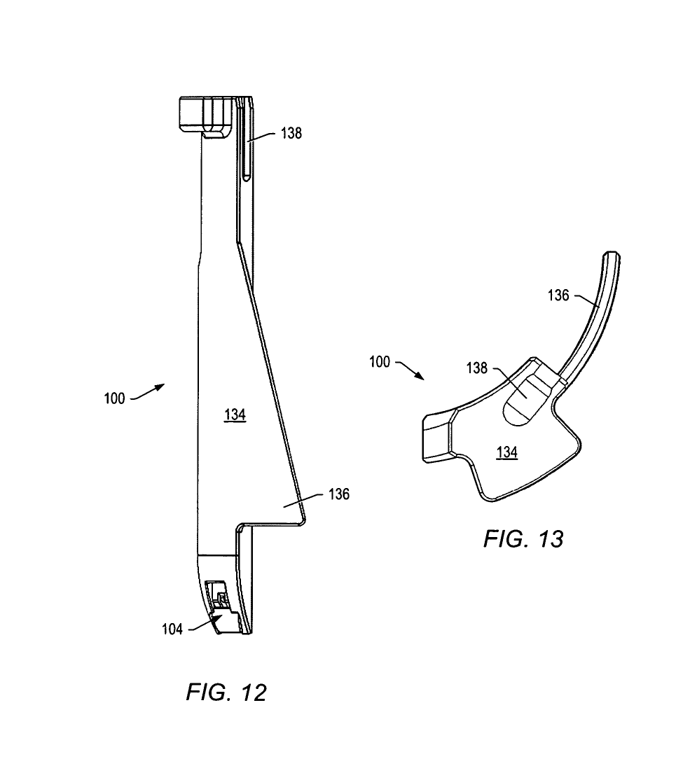

FIG. 12 depicts a front view of an embodiment of second blade 100 of a blade assembly. FIG. 13 depicts a top view of an embodiment of second blade 100. The third blade may be a mirror image of second blade 100. Second blade may include body 134, recess 138, extension 136, and extender 104. An upper portion of body 134 may reside in the second blade recess of a blade holder. An arm of the first blade may be positioned in recess 138 to couple the first blade to the second blade.

In the illustrated embodiment, the extension of the second blade may overhang one of the recessed areas of the first blade when the blade assembly is coupled to a blade holder. The extension of the third blade may overhang the other recessed area of the first blade. If the cam activator is rotated to advance the cam against the cam surface of the first blade to rotate the first blade, the first blade rotates and pushes against the extension of the second blade and the extension of the third blade. The force applied to the second blade and the third blade by the first blade rotates the second blade and the third blade and causes the distal ends of the second blade and the third blade to be spaced away from the distal end of the first blade.

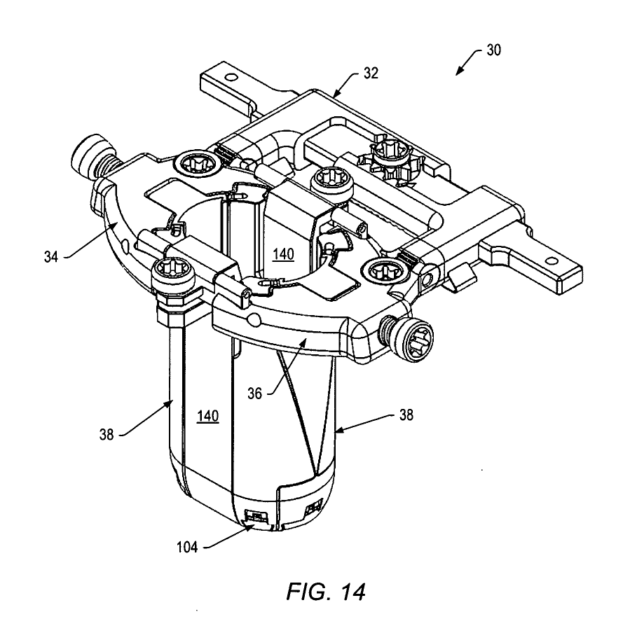

FIG. 14 depicts an embodiment of surgical retractor 30 using side blades 140. Blade assemblies 38 are positioned in blade holders 34, 36. Separator 32 has been used to separate the blade holders a sufficient distance to allow side blades 140 to be inserted into side blade recesses of blade holders 34, 36. Note as illustrated, blade assemblies are in a first or un-rotated position. Blade assemblies are not rotated relative to blade holders 34, 36. The extender bodies of the extenders 104 are retracted into the blade bodies of blade assemblies 38.

In contrast, FIG. 1 depicts surgical retractor after cam activators 74 have been used to rotate first blades 98 of blade assemblies 38. Also, a blade adjustor (not shown) has been used on extenders 104 to push portions of extender bodies out of the blade bodies of the blade assemblies.

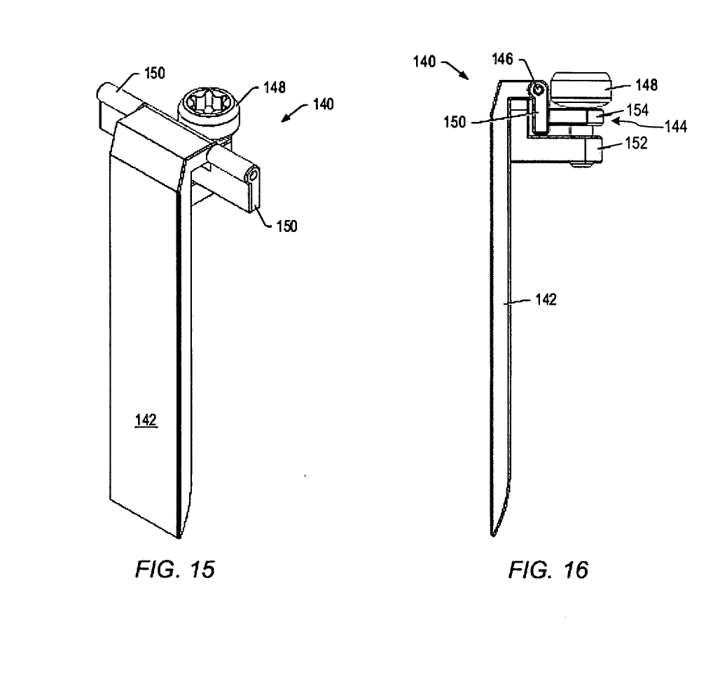

FIG. 15 depicts a perspective view of an embodiment of side blade 140 for a surgical retractor. FIG. 16 depicts a side view of an embodiment of side blade 140. In some embodiments, side blade 140 may not be positioned in the side blade recesses in the blade holders until the blade holders are separated by a predetermined distance, for instance, 30 mm. In other embodiments, the size of the side blades may be adjusted to accommodate other minimum separation distances of the blades holders.

Side blade 140 may include body 142, mount 144, pin 146, and activator 148. Pin 146 may couple body 142 to mount 144 and allow the body to rotate relative to the mount. Arms 150 of mount 144 may be positioned in the side blade recesses of the blade holders. Activator 148 may be coupled to mount 144 and to body 142. Activator 148 may include male threading that complements female threading in base 152 of body 142. When arms 150 are positioned in the side blade recesses of the blade holders, rotation of activator in a clockwise direction may draw base 152 of body 142 towards base 154 of mount 144. Body 142 may rotate so that a distal end of side blade 140 moves outwards from the blade holders. An upper surface of side blade 140 may be etched, include a decal, or include printing that indicates a direction of rotation of activator 148 to tilt the side blade. In some embodiments, rotation of activator allows side blade to tilt up to about 15° from vertical. In other embodiments, rotation of activator may allow side blade to in a range that is greater or less than 15° from vertical.

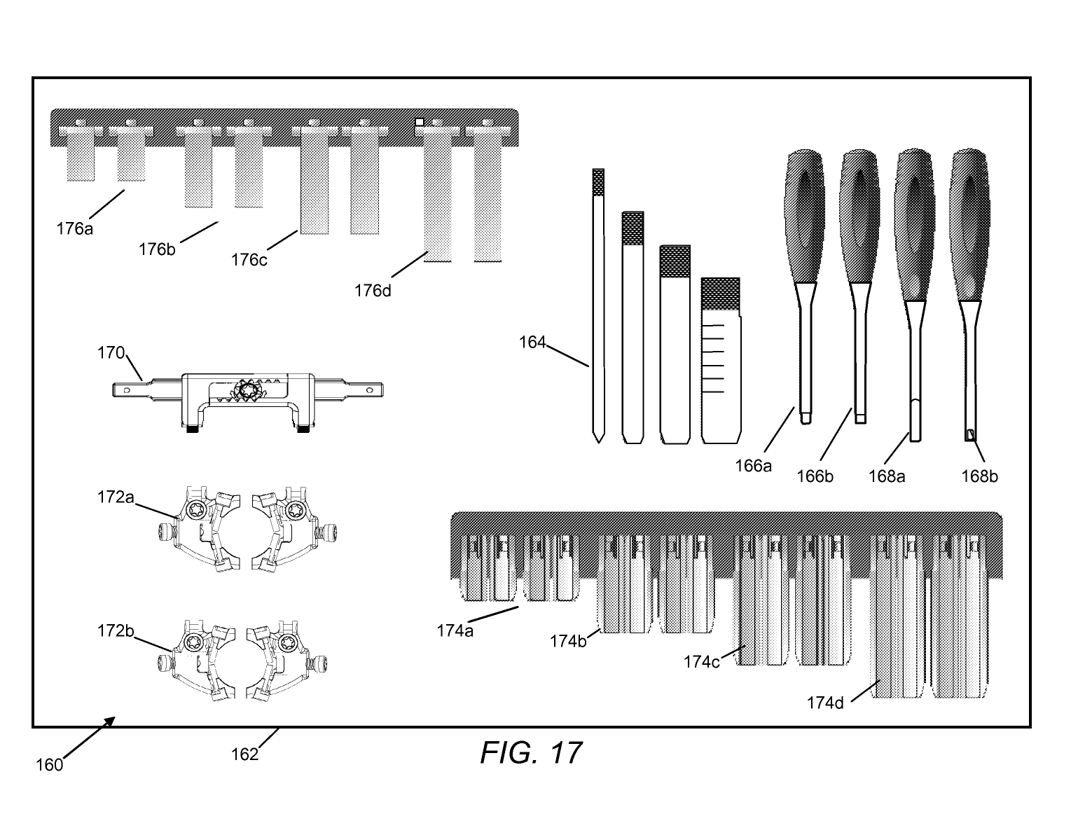

The surgical retractor may be provided in a kit 160 as illustrated in FIG. 17. The kit 160 may include one or more cases 162 that holds accessories, instruments, and the components of the surgical retractor. The cases or case 162 may have a plurality of openings. In certain embodiments, the entire case may be placed in a sterilizer to sterilize all of the contents within the case. Some of the contents in the case may be pre-sterilized and placed in bags that are put into the case. Accessories included in the case may include MediFlex Arm and Table Mount (Mediflex Surgical Products, Islandia, N.Y.), a table adaptor, light cables and adaptors, disposable light mats, and trays.

In some embodiments, instruments included in the case may include a dilator set 164, a pair of drivers 166a-166b, blade adjusters 168a-168b, one or more blade holders 172a-172b, and a connector 170 for the blade holders 168a-168b. In certain embodiments, the dilator set 164 may be used to expand the initial incision made in the patient. The drivers 166a-166b may turn the cam activators, side blade activators, angle locks and/or the pinion of the separator or connector 170. A pair of drivers 166a-166b may be included so that the same task can be simultaneously performed on the first and second blade holders of the surgical retractor. Handles of the drivers may be ratcheting or non-ratcheting handles. The retraction device may be used to draw tissue out of the way when side blades are installed in the surgical retractor. The connector 170 may join the blade holders 172a-172b together so that the same amount of rotation relative to the separator may be simultaneously applied to both blade holders. The blade adjustor may be used to move the extenders of the blade assemblies.

In some embodiments, the surgical retractor components may include the separator 170; two or more blade holders 172a and 172b, two or more blade assemblies 174a-174d, and two or more side blades 176a-176d. If the surgical retractor is to be used for a spinal procedure, the kit may include a number of pairs of blade holder and blade assembly combinations, or a number of pairs of blade assemblies, of various lengths. For transforaminal or posterior lumbar approaches, the pairs of blade assemblies included in the kit may have lengths of 40 mm, 50 mm, 60 mm, 70 mm, and 80 mm. For extreme lateral lumbar approaches (e.g., an XLIF procedure), the pairs of blade assemblies included in the kit may have lengths of 90 mm, 100 mm, 110 mm, 120 mm, and 130 mm. Other sizes and quantities may also be included in the kit.

Prior to or during the surgical procedure, blade assemblies may be placed in the blade holders, and the blade holders may be coupled to the separator. The separator may be adjusted so that the blade holders are close together. The surgical retractor may be coupled to a table mount. The drivers may be used to turn the rotation locks so that the blade holders are in an unlocked position that allows for rotation of the blade holders and blade assemblies relative to the separator.

An incision may be formed in the patient. The incision may be expanded using the dilators. When the blade holders of the surgical retractor are in an initial close together position, the blades assemblies may closely match the outside diameter of the largest dilator so that the blade assemblies may be guided into the patient along the outside surface of the largest dilator. The surgeon or operating theater personnel may grasp the blade holders and position the blade assemblies in the incision. The table mount may be locked in position. The drivers may be used to turn the rotation locks to fix the position of the blade holders and blade assemblies relative to the separator. A lighting strip may be coupled to a first blade of a blade assembly, or lighting strips may be coupled to each blade assembly. The driver may be used to turn the pinion of the separator to move the blade assemblies away from each other and retract tissue. When the blade holders are a desired distance apart, arms of side blades may be positioned in side blade recesses.

During some procedures, the driver or a driver may be used to rotate a cam activator or both cam activators of the blade holders. Rotating a cam activator of a blade holder rotates a first blade of the blade assembly coupled to the blade holder and tilts the blades of the blade assembly to achieve additional tissue retraction. Tilting the blades of the blade assembly moves the distal ends of the blades of the blade assembly outwards creating a larger opening near a distal end of the blade assembly. After the blades of a blade assembly are tilted, one or more extenders of the blade assembly may be moved outwards with the blade adjustor.

In some embodiments, the drivers or a driver may be used to rotate an activator of a side blade or the activators of the side blades. Rotating an activator of a side blade may tilt the side blade so that the distal end of the side blade moves outwards.

After the surgical retractor is positioned and set up, the surgical procedure may be performed. After the surgical procedure, a driver may be used to rotate the pinion so that the blade holders and blade assemblies are drawn close together. The surgical retractor may then be removed from the patient.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A surgical retractor, comprising:
a first blade holder coupled to a first blade activator;
a second blade holder coupled to a second blade activator;
a separator slidingly coupled to the first blade holder and the second blade holder, the separator configured to move the first blade holder relative to the second blade holder;
a first blade assembly coupled to the first blade holder, the first blade assembly including:
a first main blade having a distal and proximal portion and a first pivot axis, wherein the proximal portion of the first main blade is rotatingly coupled to the first blade holder;
a first secondary blade having a distal and proximal portion, wherein the proximal portion of the first main blade is coupled to the proximal portion of the first slide blade such that when the first main blade is rotated about the first pivot axis, the distal portion of the first secondary blade also moves;
wherein the first blade activator is moveable from a first position to a second position such that the first blade activator causes the first main blade to rotate about the first pivot axis; and
a second blade assembly coupled to the second blade holder; the second blade assembly including:
a second main blade having a distal and proximal portion and a second pivot axis; wherein the proximal portion of the second main blade is rotatingly coupled to the second blade holder;
a second secondary blade having a distal and proximal portion, wherein the proximal portion of the second main blade is coupled to the proximal portion of the second slide blade such that when the second main blade is rotated about the second pivot axis, the distal portion of the second secondary blade also moves; and
wherein the second blade activator is moveable from a first position to a second position such that the second blade activator causes the second main blade to rotate about the second pivot axis.

2. The surgical retractor of claim 1, wherein
the first blade assembly includes a third secondary blade having a distal and proximal portion, wherein the proximal portion of the first main blade is coupled to the proximal portion of the third slide blade such that when the first main blade is rotated about the first pivot axis, the distal portion of the third secondary blade also moves; and
the second blade assembly includes a fourth secondary blade having a distal and proximal portion, wherein the proximal portion of the second main blade is coupled to the proximal portion of the fourth slide blade such that when the second main blade is rotated about the second pivot axis, the distal portion of the fourth secondary blade also moves.

3. The surgical retractor of claim 1, wherein the separator comprises a bar, a first rack coupled to the bar, a second rack coupled to the bar, and a gear coupled to the bar, the first rack and the second rack; wherein gear is configured to move the first rack relative to the second rack to move first blade holder relative to the second blade holder.

4. The surgical retractor of claim 3, wherein the separator includes a stop coupled to the gear and removably coupled to the first rack, and where movement of the first blade holder relative to the second blade holder is inhibited until the stop is decoupled from the first rack.

5. The surgical retractor of claim 1, wherein the first blade holder is removably coupled to the separator, and the first blade assembly is affixed to the first blade holder.

6. The surgical retractor of claim 1, wherein the first blade holder is affixed to the separator, and the first blade assembly is removably coupled first blade holder.

7. The surgical retractor of claim 1, wherein the distal portion of at least one of the blades is slidingly coupled to a blade extension.

8. The surgical retractor of claim 1, wherein at least one of the blades include a longitudinal slot and a light mat position within the slot wherein further comprising an illumination source coupled to the first blade assembly.

9. The surgical retractor of claim 1, further comprising:
a first recess positioned within the first blade holder;
a second recess positioned within the second blade holder; and
a side blade having a fixed portion coupled to the first recess and the second recess and a rotating portion rotatingly coupled to the fixed portion.

10. A method of retraction during surgery, comprising:
slidingly separating a first blade holder from a second blade holder, wherein the first blade holder includes a first blade assembly and the second blade holder includes a second blade assembly;
rotating a first main blade of the first blade assembly to tilt with respect to the first blade holder such that the first main blade moves a first secondary blade; and
rotating a second main blade of the second blade assembly to tilt with respect to the second blade holder such that the second main blade moves a second secondary blade.

11. The method of claim 10, wherein the slidingly separating comprises disengaging a round gear from a stop such that the gear engages a first plurality of teeth coupled to the first blade holder and a second plurality of teeth coupled to the second blade holder such that first blade holder moves with respect to the second blade holder.

12. The method of claim 10 wherein the rotating a first main blade further comprises moving a third side blade.

13. The method of claim 10 wherein the rotating a second main blade further comprises moving a fourth side blade.

14. The method of claim 10, further comprising inserting one or more side blades in the surgical retractor.

15. The method of claim 10, further comprising illuminating an area between the first main blade and the second main blade with a light mat coupled to at least one of the first main blade or second main blade.

16. The method of claim 10, further comprising extending a length of at least a portion of the first blade assembly after rotating the first blade of the first blade assembly.

17. A kit, comprising:
a separator;
a first pair of blade holders having a first pair of arms with a first predetermined length, wherein the first pair of arms are configured to couple to the separator;
a second pair of blade holders having a second pair of arms with a second predetermined length, wherein the second pair of arms are configured to couple to the separator;
a plurality of pairs of blade assemblies configured to couple to the blade holders, wherein each pair of blade assemblies has blades of differing lengths; and
at least one driver configured to activate moving components of the separator and the blade holders.

18. The kit of claim 17, further comprising at least a pair of side blades.

19. The kit of claim 17, further comprising a blade adjustor configured to lengthen a portion of a blade assembly.

20. The kit of claim 17, further comprising at least one illumination source configured to couple to a blade assembly and a dilator set.
